(19) United States
(12) Patent Application Publication
(10) Pub. No.: US 2023/0343470 A1
(43) Pub. Date: Oct. 26, 2023

(54) Infection Risk Simulation and Control System

(71) Applicant: Stephen A. Yencho, Rowlett, TX (US)

(72) Inventor: Stephen A. Yencho, Rowlett, TX (US)

(21) Appl. No.: 17/913,809

(22) PCT Filed: Mar. 26, 2021
(86) PCT No.: PCT/US2021/024434

§ 371 Date: Sep. 22, 2022

Related U.S. Application Data
(60) Provisional application No. 62/994,953, filed on Mar. 26, 2020.

(51) Int. Cl. G16H 50/80; A61L 9/20; G06F 3/01

(52) U.S. Cl. CPC G16H 50/80; A61L 9/20; G06F 3/011; A61L 2209/111; A61L 2209/16

(57) ABSTRACT

A method for airborne pathogen disinfection and pathogen risk determination in near real-time in an indoor area that may hold at least one occupant may include providing a pathogen risk simulation system; inputting at least one generally-fixed quantity associated with the indoor area to the pathogen risk simulation system; sensing, with the pathogen risk simulation system, at least one quantity associated with the indoor area; and based on the inputting and the sensing, computing pathogen density and risk level in the indoor area. A simulation system for determining pathogen risk in an indoor area may include a central processing unit; at least one occupancy sensor connected to the central processing unit; at least one air disinfection system interface connected to the central processing unit; and a display connected to the central processing unit.

### CROSS-REFERENCE TO RELATED APPLICATIONS

This application claims the benefit of priority to U.S. Provisional Application No. 62/994,953, filed Mar. 26, 2020, which is hereby incorporated by reference in its entirety.

### FIELD OF THE INVENTION

The invention generally relates to methods of controlling and monitoring pathogen exposure level in a space, and more specifically to a method of disinfecting airborne pathogens and monitoring pathogen exposure by modeling exposure in near real-time and displaying near real-time pathogen risk information.

### BACKGROUND

Prophylactic vaccines have been developed for many viruses, though some vaccines may not completely prevent disease transmission. Even after full vaccination of a target population, substantial disease transmission risk may continue. Mutant viral strains may evade the protection of vaccines, further increasing risk of disease transmission. Therefore, substantial infection risk may remain for some viral pathogens as groups of people gather, especially in indoor spaces. Viral pathogens are transmitted though airborne droplets and aerosols as well as by surface contact. Viral aerosols may remain suspended in air for hours, further increasing disease transmission risk. The risk of transmission and infection increases as groups of people increase in size. As of the filing date of this document, the virus referred to as SARS-CoV-2, which causes the COVID-19 infection, is a viral pathogen of particular concern not only in the United States, but also the entire world.

Major challenges exist in rapidly and accurately testing surfaces and air for pathogens. Current technology to determine pathogen presence on surfaces and in air relies upon swabbing surfaces to acquire a sample which is usually incubated for 24 hours or more to test for the presence of a pathogen. Alternately, Polymerase Chain Reaction (PCR) tests may be used to test air samples. Alternately, Rapid Adenosine Triphosphate (ATP) tests may also be used for testing of surfaces and air. However, neither PCR nor ATP tests can distinguish between viable and non-viable virus particles. Both viable and non-viable virus may be present in room air or on surfaces, especially when ultraviolet (UV) or far-ultraviolet disinfection systems are used. All known tests for viruses and pathogens take time to provide results; it is impossible, using current technology, to detect a virus or to detect virus concentration in room air or on surfaces in near real-time. As used in this document, the term “near real-time” refers to a period of time that it takes a program loop to execute, which is a time period on the order of several seconds to several milliseconds. “Near real-time” does not include periods of time of thirty seconds or more.

### SUMMARY OF THE INVENTION

A method for determining pathogen risk in near real-time in an indoor area that may hold at least one occupant may include providing a pathogen risk simulation system; inputting at least one generally-fixed quantity associated with the indoor area to the pathogen risk simulation system; sensing, with the pathogen risk simulation system, at least one quantity associated with the indoor area; and based on the inputting and the sensing, computing pathogen density and risk level in the indoor area.

A method for determining pathogen risk in an indoor area may include activating a pathogen risk simulation system; activating sensors in the indoor area associated with the pathogen risk simulation system; receiving input from the sensors at the pathogen risk simulation system; based on the inputs, determining pathogen risk within the indoor area with the pathogen risk simulation system; and communicating the pathogen risk, with the pathogen risk simulation system, to one or more users.

A simulation system for determining pathogen risk in an indoor area may include a central processing unit; at least one occupancy sensor connected to the central processing unit; at least one air disinfection system interface connected to the central processing unit; and a display connected to the central processing unit.

The present invention addresses these challenges through an infection risk simulation system which can provide immediate feedback on pathogenic risk not available from conventional methods. The system includes a method for computing and displaying the results of a pathogen transmission simulation model in real-time or near real-time based on current and prior room conditions and current and prior occupancy levels. The simulation model may optionally be used to control building heating, ventilation, and air conditioning systems (HVAC), air disinfection, and other pathogen control systems. Alternately, the system may be used to calibrate existing building control systems and optimize their performance with air and surface disinfection systems. Sensors on the simulation system may be replaced with simulated inputs to obtain an iterative algorithm for optimizing the safety of occupied buildings and occupied spaces in addition to or in place of physical testing.

With proper calibration and accurate simulations, the pathogen risk simulation system can provide a rapid and accurate estimate of pathogen risk, which is not possible using technology available today. The method can provide immediate feedback of pathogen risk to occupants of the space, avoiding the high cost and lengthy delays from conventional pathogen testing. The present invention solves the problem of detecting a virus or detecting virus concentration in room air or on surfaces in near real-time, by combining known quantities and controlled quantities in a particular location with scientifically-validated assumptions to generate a simulation of viral or pathogen concentration that is usefully close to the actual viral or pathogen concentration.

Calibration of the simulation may be done through actual sampling of surfaces and air or optionally by DNA air and surface transmission studies to quantify air flow or through carbon dioxide studies. Alternately, computational fluid dynamics simulations or another suitable method may be used. Optionally, in addition to pathogens, risks due to dust, pollen, mold spores, chemical exposure, and even radioactive particles in the air and on surfaces may also be simulated.

Although the infectious dose of a pathogen may vary from person to person, scientific research has established approximate infectious dosage levels for common pathogens which may be used in a computational model. Inferring pathogen infection risk though use of a calibrated computational model preferably includes all relevant factors to compute a risk level in near real-time for an occupied area or sub-area of an occupied area. Alternately, the system may use inputs from pathogen air and surface measurement systems such as mass spectrometers or other analytical instruments.

Substantial costs may be incurred to equip indoor spaces to provide pathogen safe air, especially at maximum occupancy. These costs may be substantially reduced by real-time pathogen risk simulation. The Centers for Disease Control (CDC) recommends at least 12 air changes per hour to reduce the pathogen transmission risk level of indoor air. The European Union recommends at least 15 air changes per hour. Most existing commercial and residential HVAC systems are not designed for this rate of air exchange. Furthermore, the American Society of Heating, Refrigeration, and Air Conditioning Engineers (ASHRAE) has recently provided guidance that existing HVAC systems alone cannot sufficiently remove viruses from indoor air, though HVAC systems can sufficiently remove bacteria, pollen, dust, and mold spores.

Compliance with rigorous building energy codes in the United States and in other countries may prevent a high percentage of pathogen free outside air from being blended into a building's recirculated air due to the energy required to heat or cool, and possibly humidify or dehumidify, the outside air.

The nanoscopic size of viruses requires HEPA or MERV 13 or higher filter or coated HEPA or other media can be added to heating, ventilation, and air conditioning (HVAC) systems to remove viruses from air. However, adding these high efficiency filters to HVAC systems may interfere with proper system operation. Such high efficiency filters may cause excessive air flow restriction or pressure drop in the system.

Air disinfection and high efficiency filtration products can substantially prevent airborne disease transmission, though the cost of these systems increases substantially as the volume of air purified increases. A wide range of pathogen disinfection technologies can be applied to augment HVAC systems and disinfect air in spaces where people gather, including air purifiers, far UV and upper air UV disinfection systems.

A reservoir of safe air can exist in an occupiable indoor or indoor/outdoor space that has been unoccupied or partially occupied for a sufficient amount of time for the suspended pathogens to settle out or be flushed out by air circulation, or in a space that has substantial volume in addition to he occupied area, such as an auditorium with a high ceiling. Such a “safe air reservoir” exists in nearly all indoor public spaces after aerosols and droplets sink to the floor over time. This safe air reservoir is included in the computed pathogen risk simulation model described below.

In addition to simulating the pathogen risk, the system may monitor performance of air disinfection and air movement systems and optionally control those systems by controlling fan speeds, disinfection systems, etc. Optionally, it may be integrated into building control systems. Optionally, it may include interfaces to a variety of building control systems and air disinfection systems and enable the user to select one or more specific models from a menu of commercially available units.

### BRIEF DESCRIPTION OF THE DRAWINGS

FIG. 1 is a cross-sectional perspective view of an air disinfection system.

FIG. 2 is a perspective view of the air disinfection system of FIG. 1.

FIG. 3 is a perspective view of air streamlines during operation of the air disinfection system.

FIG. 4 is a block diagram of the air disinfection unit controls and interfaces.

FIG. 5 is a block diagram of a room pathogen safety indicator.

FIG. 6 is a block diagram of a programmable switch for a UVC/visible light lamp.

FIG. 7 is a partial cross-sectional perspective view of a trackable wipe.

FIG. 8 is an perspective view of an occupied single room with a pathogen risk simulation system.

FIG. 9 is a block diagram of an integrated pathogen risk simulation system with wireless sensors.

FIG. 10 is a block diagram of a remotely hosted pathogen risk simulation system.

FIG. 11 is a simplified software algorithm flow chart.

FIG. 12 is a perspective view of an occupied space with multiple zones.

The use of the same reference symbols in different figures indicates similar or identical items.

### DETAILED DESCRIPTION

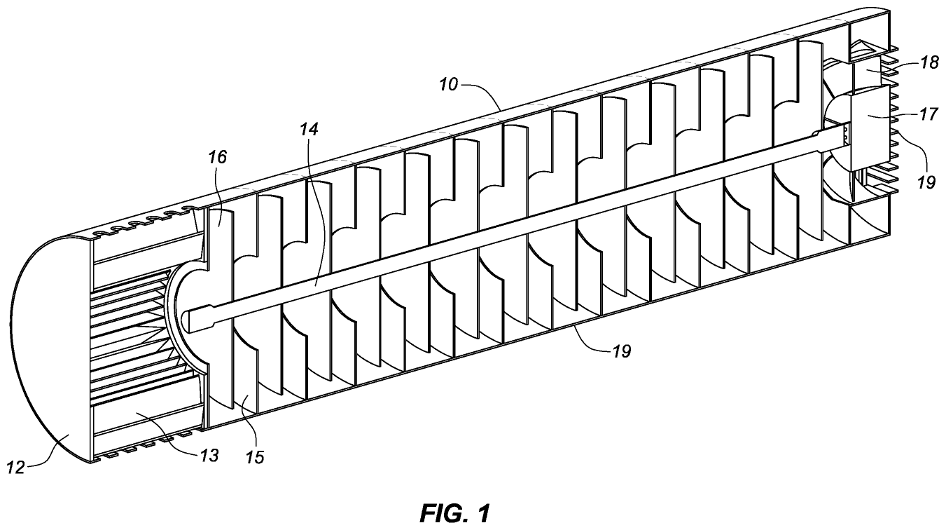

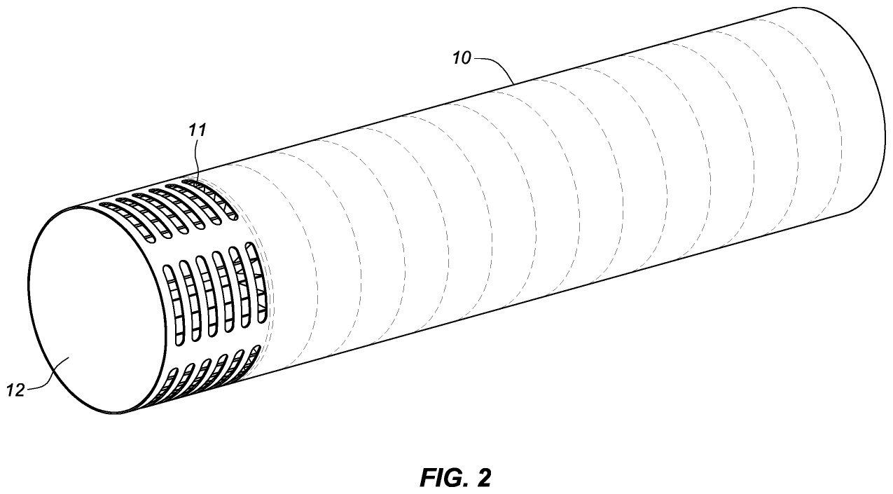

The air disinfection system shown in FIG. 1 and FIG. 2 is designed to disinfect fluids with low viscosities, such as air and a variety of gases. The disinfection system preferably consists of alternating annular baffles 15 and disc baffles 16 spaced apart along a centrally located UVC light source 14. Alternately, the lamp may be off center. Alternately, multiple lamps may be used. Alternately or in addition, lamps may be arrayed around the outside of the chamber. Alternately, the chamber wall 19 may be made of UVC-transparent material such as fused quartz or fluoroethylene propylene (FEP).

In the chamber 10, annular baffles 15 alternate with disc baffles 16. The space between successive annular baffles may be referred to as a subchamber. Preferably the baffles are equally spaced apart along the chamber axis. Alternately, the spacing may vary. Alternately, the chamber may have an oval, square or polygonal cross-sectional shape. Alternately, the chamber may have any other suitable shape.

Preferably, chamber 10 and baffles 15 and 16 are fabricated from aluminum alloy, which reflects up to 98% of UVC light. Alternately, they may be made of another material. Alternately, they may be coated with aluminum. Alternately, they may be stainless steel. Alternately, they may be coated with silver. Alternately, they may be made from another suitable material. Preferably, a series of longitudinal strips located along the baffle edges inside the chamber wall 19 are used to mount the baffles to wall of chamber. Alternately, another mounting technique is used.

Disc baffle 16 preferably has a center hole to accommodate light source 14. The hole in disc baffle 16 preferably is preferably within a 3 mm radial distance to light source 14. Alternately, it may be any other suitable distance. Alternately, the baffles may be of different design. Optionally the baffles and/or baffle openings may incorporate serrated edges, flanges on the edges or on planar surface and/or vortex generators or fins. Optionally, the baffles may not be planar. Optionally, the baffles may include features to induce fluid swirling or fluid tumbling.

Preferably, ballast-driven low pressure mercury UVC lamps are used as light sources 14. Alternately, microwave powered UVC low pressure mercury lamps may be used. Alternately, high pressure mercury lamps may be used. Alternately, UVC LEDs may be used. Alternately, another light frequency such as far UV (200 nm to 220 nm) may be used. In the preferred embodiment, the lamp does not produce ozone by using a lamp or surrounding borosilicate glass tube which filters out ozone causing wavelengths of ultraviolet light. Alternately, the lamp may be configured to produce ozone. Optionally, a fused quartz tube or an FEP tube or shield over the lamp or LEDs may be used. Optionally, heating elements may be added to system 10 to increase the temperature of the air passing through the chamber, thus increasing the inactivation rate of the airborne pathogens. Alternately, system 10 may not employ UVC light to inactivate the airborne pathogens and may instead use heat to kill the airborne pathogens.

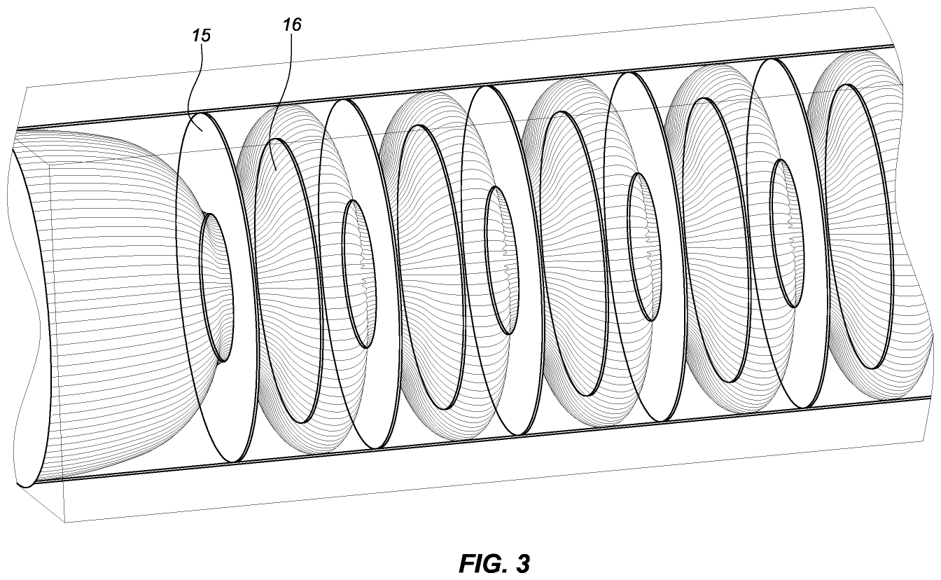

During system operation, air enters air disinfection unit 10 through intake passages 11 and then passes through air filter 13. Air filter 13 is preferably a HEPA filter to remove at least 99.97% of airborne particles 0.3 micrometers (μm) in diameter to prevent airborne dust, allergens, some pathogens, and other contaminants from entering the disinfection unit. Alternately, another type of air filter may be used. End cap 12 restricts the air flow to move through the central opening in the first of annular baffles 15. Air then enters a series of chambers by passing through the annulus in annular disc 15. There is a gap between the chamber wall 19 and at least part of the outer edge of the disc baffle 16. In this way, air is forced by each disc baffle 16 to flow radially outward prior to air motion longitudinally, and then air is forced by the inner surface of the chamber wall 19 to flow radially inward as shown by streamlines of an exemplary set of subchambers in FIG. 3. This cycle repeats until the fluid has passed through all of the subchambers.

As the air passes thru a succession of these subchambers, the viral aerosols and other pathogens in the air are dimerized by the UVC light, thus inactivating them and disinfecting the air. The series of subchambers acts to limit the progression of micro currents of air which can advance ahead of bulk airflow in a spiral chamber or a tubular UV disinfection chamber, thus preventing viable virions and other pathogens from passing through the disinfection chamber without being inactivated. The system is intended to maximize the pathogen inactivation rate while minimizing the pressure drop along the system. Therefore, the present invention is able to achieve a given level of disinfection in a smaller volume with minimal UVC energy and minimal chamber volume to enable it to be used as a practical solution.

After passing through the succession of subchambers, the flow is urged through exhaust louvers 18 by exhaust fan 17. Negative pressure from the exhaust fan 17 also pulls air into the intake passages 11. Preferably, exhaust fan 17 is an axial flow fan. Alternately, another type of exhaust fan is used. Alternately, a fan may be placed at the inlet of the system to propel air through the system. Alternately, multiple fans may be employed. Alternately, fans may be placed in series between the subchambers. Alternately, a pressure difference on the inlet and outlet of the chamber is used to draw to propel air through the system. Alternately, one or more intermediate pressure differences on the chamber is used to draw to propel air through the system. Alternately, air may be propelled through the system by pulsed flow from a bellows, diaphragm, linear compressor, or other source. Alternately, radiation coupling is used to move the air. The present invention may be used to disinfect a wide range of gasses and liquids, including water and other more viscous fluids and organic fluids.

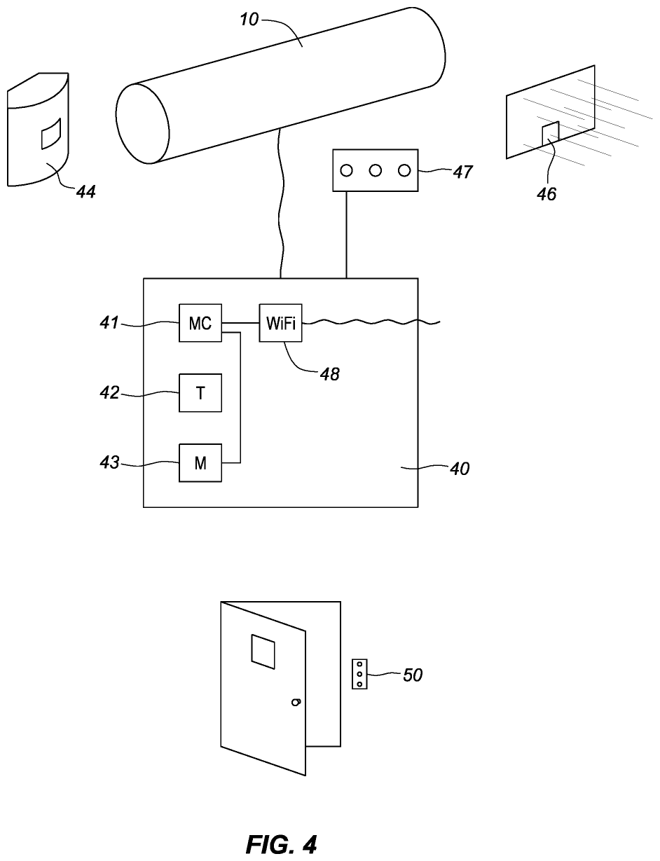

In another aspect of the invention, air disinfection system 10 may employ control unit 40 with embedded microcontroller 41 and a set of sensors as shown in FIG. 4. Control unit 40 preferably includes a room temperature sensor 42 and humidity sensor 43. It optionally includes a motion detector 44 and/or a room occupancy sensor 44. Output signals from analog sensors are digitized by converting the output signals to digital through the use of an appropriate analog to digital converter to interface to the microcontroller. Optionally, multiple sensors of each type may be used to create redundancy to compensate for drift, noise, or sensor failure. Optionally, interfaces such as I2C or SPI or any other suitable interface may be used to interface to sensors with the appropriate output interface.

Control system 40 optionally incorporates one or more flow sensors 46 to sense airflow from existing HVAC duct(s) into the room. The flow sensors 46 may be positioned within the ducts and/or at the exit or exits of the ducts. The control system 40 may be connected to an air disinfection unit 10. Optionally, control system 40 is interfaced to existing HVAC systems. Control system 40 may employ an algorithm to compute the time needed to properly disinfect the room air based on the number of room air changes per hour. Research has shown that increased numbers of room air changes per hour substantially reduce the risk of disease transmission from viral aerosols. Because the system of the present invention disinfects the air in the room, fewer numbers of air changes per hour may be required to maintain pathogen safe air. For proper air disinfection calibration, controller 40 requires input of the room air volume. Based on the room size, the number of air changes per hour may be computed by the controller 40 and this information is then used to compute and display the airborne pathogen safety level. According to some embodiments, the controller 40 may directly control the fan speed or other factor relating to airflow in an existing HVAC system, thereby controlling the number of air changes per hour. A configuration website or cell phone application program may be used to configure and transmit this information and other required information to control system 40. Alternately, a set of switches or jumpers on control system 40, or other inputs and/or displays, may be used to set the room size and other parameters.

Machine learning statistical techniques may be used to further optimize the algorithm over time based on air samples acquired from the room. The airborne pathogen safety level for the room may be displayed on the air disinfection unit through the use of a status display or discrete LEDs 47 mounted on the air disinfection unit 10 and/or by wireless communication link 48 to a room pathogen safety indicator outside the room or at any other suitable location. Optionally, control system 40 can wirelessly transmit the room pathogen safety level and other operational information to cellular phones or to a database through wireless data transfer. The airborne pathogen status information may optionally be transmitted to a remote computer for processing and/or stored in a remote database for analysis by statistical quality control techniques or other purposes.

Optionally, the database may be configured to allow access from authorized personnel to manually update the pathogen safety status of rooms as well as accept automatic updates from control system 40 or other pathogen safety control systems. The pathogen safety status of public places such as hotels and restaurants may be updated manually as well. A smartphone application program may provide display of the pathogen safety status of a wide range of rooms or other places for a broad range of users to see. Optionally, the pathogen safety status may be displayed as a map of a geographic area with colors corresponding to the pathogen safety level of a particular place, including entire buildings and even cities and regions.

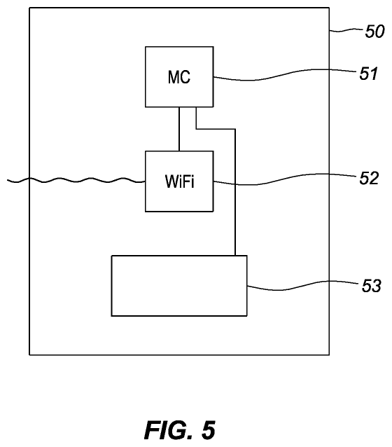

The room pathogen safety system 50 as shown by the block diagram in FIG. 5 displays room pathogen safety status from the air disinfection unit 10. Pathogen safety system 50 preferably incorporates a microcontroller 51 with a wireless receiver 52 to receive data to display results on display 53 from an algorithm running internally or remotely. Preferably, system 50 has wireless receiver 51 to receive signals from room disinfection systems such as air disinfection systems and surface disinfection systems. The status display may be an LED screen, and LCD screen or discrete multi-colored LEDs. Each lit LED would correspond to a room pathogen safety level, such as green for safe, yellow for not fully safe, and red for unsafe.

Optionally, system 50 may display combined data from multiple disinfection systems such as room air disinfection system 10, ultraviolet surface disinfection systems, surface disinfection system 80, whole room UVC disinfection systems, floor disinfection systems, other systems, and manual inputs. The disinfection results from these multiple disinfection systems are collectively input into a disinfection simulation model which sums the disinfection effects from all these devices for their respective surfaces and the air (and possibly the faucet, shower, and toilet water) to derive a composite disinfection score for the room. For example, each surface disinfection tracking system provides data on the disinfection log kill rate across the specific surfaces which were disinfected. This data may be overlaid with disinfection simulation results from ray tracing computation of the UV rays from one or more whole room UV robots. The airborne pathogen disinfection data may be weighted and summed to arrive at a composite room pathogen safety level which is then displayed on a graphic or alphanumeric display or by a series of lights or LEDs which indicate safety level from green (high) to yellow (intermediate) to red (poor). Machine learning techniques may be used to optimize the results and provide guidance for improved disinfection. The results from the simulation are optionally stored in a database for a historical record and for use by a statistical quality control system.

Optionally, the disinfection simulation algorithm may run on the microcontroller of pathogen safety system 50. The algorithm preferably compensates for the effects of disinfection decay over time by reducing the room safety level accordingly as time passes until the next cleaning event(s).

Another embodiment of the present invention is a portable battery powered version that fits in a knapsack or is worn by the user. Optionally, this portable system may not require a fan. Instead, a breathing tube may be used to draw in air through the system through a hose to a face mask. A small bladder may be used to dampen surges through the system as the user inhales. Alternately, the unit may be pocket sized and be held up to mouth and breathed through by the user, instead of needing to have a hose connection to a fixed unit. In this case, the user would need to move the soft pallet in the rear of their mouths upward to close off their nasal passages or wear a nose plug. The pocket sized unit preferably has an indicator to show when the unit has self-disinfected with its internal UVC light source and is ready to take a breath from.

### Pathogen Risk Simulation System

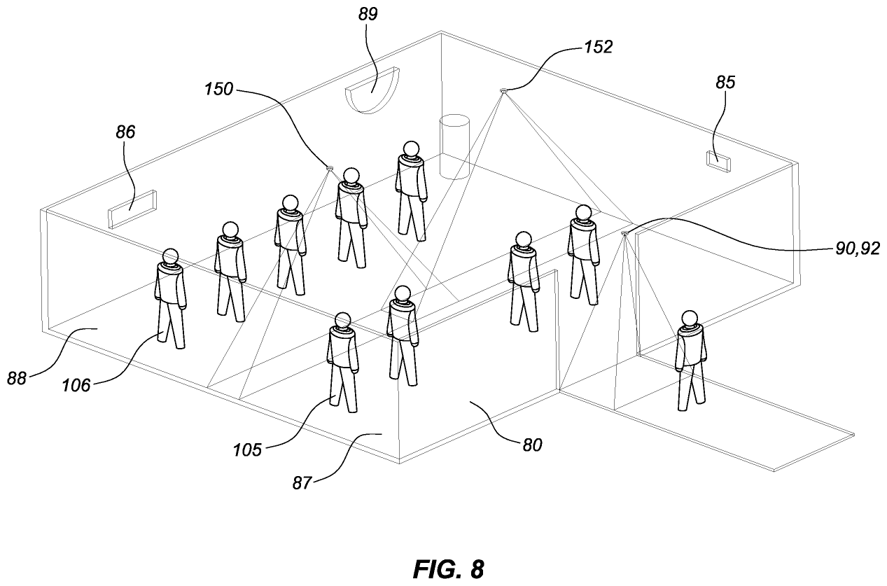

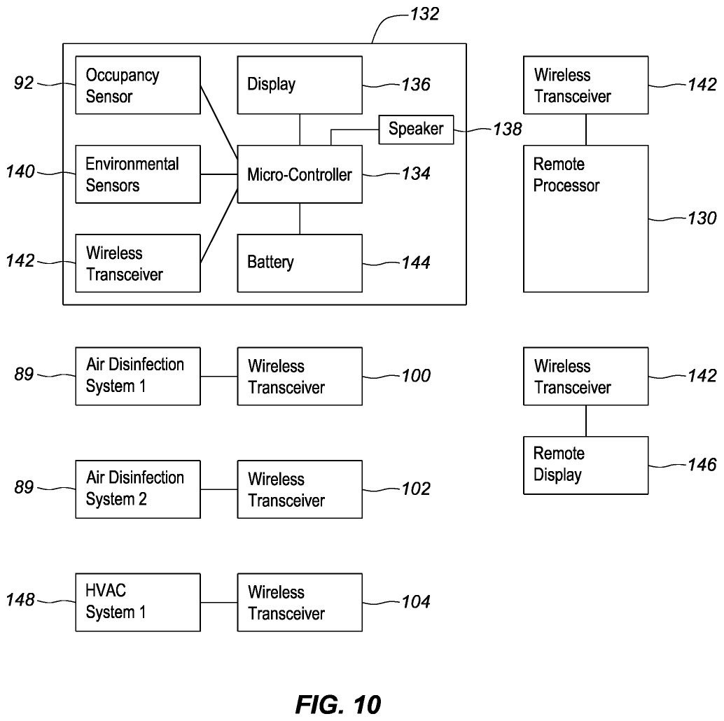

A first embodiment of an exemplary system of the present invention is shown in FIG. 8. Referring to FIGS. 8-10, another embodiment of a pathogen risk simulation system 90 is shown, installed in an occupied room 80. As used in this document, the term “room” refers to any indoor area and is not limited to any particular type of indoor area, nor is it limited to commercial or residential indoor areas. Similarly, the term “indoor area” includes any enclosed or semi-enclosed space, and excludes spaces that are not enclosed. For example, the interior of a subway car, the interior of a subway station, the inside of an automobile, the interior of a tent with generally-enclosed sides, and the interior of a building all are indoor areas. The space under an outdoor canopy with no side walls is not an indoor area, because that space is not enclosed or semi-enclosed. A standalone simulation system 90 includes at least one occupancy sensor 92 and other types of sensors which may include one or more air flow sensors (not shown), one or more temperature sensors 98, one or more humidity sensors 98, and hardware and software (described in greater detail below) with the ability to track or simulate these sensor inputs over time. As shown in FIG. 8, the standalone simulation system 90 and at least one occupancy sensor 92 may be housed in the same structure. Alternately, instead of the standalone simulation system 90, a networked system 101 consisting of one or modules is used as shown in FIG. 10.

The room 80 may include one or more air supply ducts 85 and one or more return air ducts 86. The air flow rates of each of these ducts may be known as a function of fan speed, or may be measured for inclusion in the simulation. Optionally, the fraction of outside air mixed with recycled air for air supply ducts 85 is also included in the simulation if applicable.

Optionally, one or more air disinfection systems 89 may be associated with the room 80, and may include a wide range of systems such as ultraviolet disinfection units and far ultraviolet disinfection systems. These air disinfection systems 89 may also include ionization and chemical disinfection systems such as dry hydrogen peroxide systems. The flow rate and disinfection rates of each of these air disinfection systems 89 is known for inclusion in the simulation model. The one or more air disinfection systems 89 may be located in or adjacent to the room 80, or may be outside the room 80 and utilized to treat air that travels into the room 80 via the air supply duct or ducts 85. As described in greater detail below, the one or more air disinfection systems 89 may be selectively controlled to an on or off state, or may be always on. As one example, one air disinfection system 89 that may be always on is an upper air UV light, which is a UV light that is directed upward and kills pathogens in the upper area of the room 80, such as the ceiling; the upper air UV light is directed upward so that its emission does not hurt the skin or eyes of the occupants of the room 80. As another example, one air disinfection system 89 that may be always on is a far UV light, such as an LED. Far UV light is approximately 222 nm, and does not hurt the skin or eyes below a certain emission intensity defined by the U.S. Food and Drug Administration.

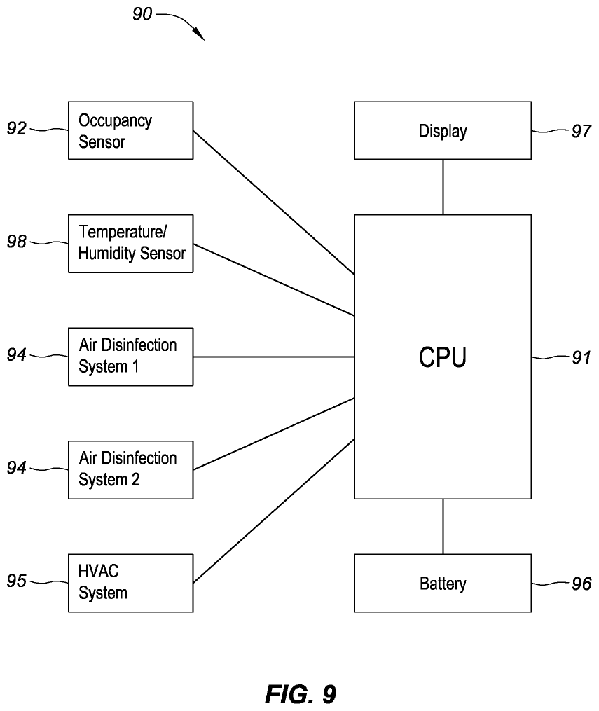

Referring also to FIG. 9, the simulation system 90 integrates these inputs. Optionally, the simulation system 90 may also tracks the performance of each of these systems. Optionally, the simulation system 90 may also control the performance of each of these systems through appropriate interfaces. The simulation system 90 optionally may be interfaced with the building control system to provide feedback to optimally reduce infectious potential of occupied rooms by air flow and outside air blend and air purification devices

The simulation system 90 includes a central processing unit (CPU) 91 with an optional display 97, thermal/humidity sensor 98, one or more air disinfection system interfaces 94 (which can monitor or control), and one or more HVAC system interfaces 95 to monitor or control the building HVAC system. Simulation system 90 may also include fans such as overhead fans or air circulation fans in room 80 (not shown). Optional battery 96 is used to power the system. Alternately, another power source is used, such as wired power in the room 80.

The occupancy sensor 92 for simulation system 90 can be of several types described below. Occupancy sensor 92 preferably measure pedestrian traffic both in and out of room 80 simultaneously. Alternately, simulated pedestrian traffic is used in lieu of actual measurements as an input for the simulation model.

Advantageously, because the system of the present invention divides the space into zones and tracks of simulates the occupants of these zones, the system of the present invention readily accommodates varying levels of occupancy or high levels of pedestrian traffic such as malls, restaurants, and other public places.

Occupancy sensors 92 may include one or more overhead cameras 90 located near each room entrance. The camera field of view preferably includes the entire walkway into room 80 in case the doorway is wide enough for people to walk into room 80 in groups or enter and exit from room 80 simultaneously. Overhead camera 90 preferably provides instantaneous occupancy levels in room 80 by tracking pixel color changes as the heads of people entering and exiting the room pass through the camera field of view.

Other examples of occupancy sensor 92 include entry/exit light beam sensors, ultrasonic sensors, Light Detection and Ranging (LIDAR) occupancy sensors, floor weight sensors, chair sensors, facial recognition cameras, thermal sensors, or sensors which measure occupancy through the measurement of CO2 exhaled in the room. The occupancy sensor or sensors 92 may be configured to sense the body temperature of the occupants of the room 80, such as by utilizing an infrared camera or other sensor. Elevated temperature of an occupant may be an input into the simulation system 90; elevated temperature of one or more occupants may be factored into the risk calculation of block 224 below. Optionally, if an occupant enters the room with an obvious fever, such as a temperature of 103° F. or higher, the simulation system 90 may generate a warning tone via the speaker 138 and/or a visual warning via the display 136. Alternately, RFID tags or wireless signals from each occupant, such as from an occupant's employee ID badge or conference attendee badge, may be tracked. An occupant's badge optionally includes other relevant information stored in association therewith, such as but not limited to the wearer's vaccination status relative to a particular pathogen.

FIG. 10 shows a block diagram of a networked multi-zone pathogen risk simulation system 90. Optionally, simulation system 90 may be remotely hosted on one or more computers or remote processors 130 not physically present at the site of room 80. The computer or remote processor 130 may be located on-site outside the room, may be a cloud computing service, or may be any other suitable computer or remote processor 130 in any suitable location. A simulation system control module 132 may be a part of the multi-zone simulation system 90, and may include a central processing unit (CPU) or microcontroller 134 connected to an optional display 136. Optionally, the system status and air safety level in each zone may be displayed through color LED displays or on smart phones or any other suitable method. A heat map display may be used to display the pathogen risk in the space on a display. Optionally, the information may be displayed in near-real time or at any other suitable interval on a smartphone or other electronic device. Audible sounds or alerts may optionally accompany the display as needed, and may be emitted by a speaker 138 connected to the microcontroller 134.

The simulation module 132 may further include a thermal/humidity sensor 140 and/or one or more additional or alternative environmental sensors 140, at least one occupancy sensor 92 as described above, and a wireless transceiver 142 to communicate with the remote processor 130. Alternately, another power source is used. Communication via the wireless transceiver 142 may occur through wireless protocols such IEEE 802.11 a/b/c/g/n (Wi-Fi), Bluetooth, or any other suitable wireless method. Alternately, communication may occur wirelessly through optical links. Alternately, at least one set of communications may occur through wired links rather than through the wireless transceiver 142 of the simulation module 132. A battery 144 may be used to power the simulation module 132.

Referring to FIG. 10, one or more air disinfection systems 89 may be interfaced to the system to allow for monitoring or control. One or more HVAC system interfaces 148 may monitor or control the building HVAC system. Simulation system 90 may also include fans such as overhead fans or air circulation fans in room 80 (not shown). An optional remote display 146 may be used, and may be connected to a wireless transceiver 142 that communicates with the wireless transceiver 142 associated with the simulation module 132.

Zones may be used to divide a single room into smaller spaces. They may also be used to divide a room with multiple entrances into zones suitable for the simulation model. Stairways and elevators may be separated into one or more zones to simplify analysis. Referring to FIG. 8, a room 80 in which the simulation system 90 is utilized may be divided into zones 87 and 88. Occupants 105 occupy zone 87. Occupants 106 occupy zone 88. Occupancy sensors 150 and 152 may continuously count occupants moving between zones 87 and 88. As used here, the term “continuously” means at spaced-apart intervals of time, across a duration of time; for example, once per second. Together with room occupancy sensor 92 at the door, near-instantaneous occupancy levels of each of zones 87 and 88 may be computed through monitoring movement within the room using occupancy sensors 84 and 85. Each zone 87, 88 may have its own unique pathogen risk simulation, as described below. Air and pathogens in the room 80 may be computed to flow within and between zones 87, 88 using the simulation algorithm.

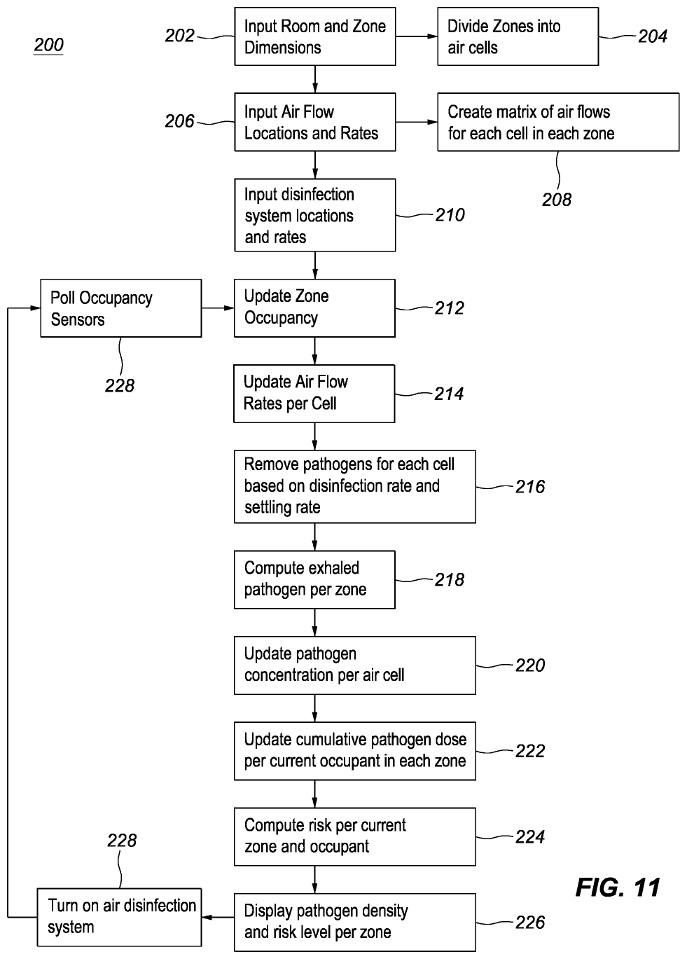

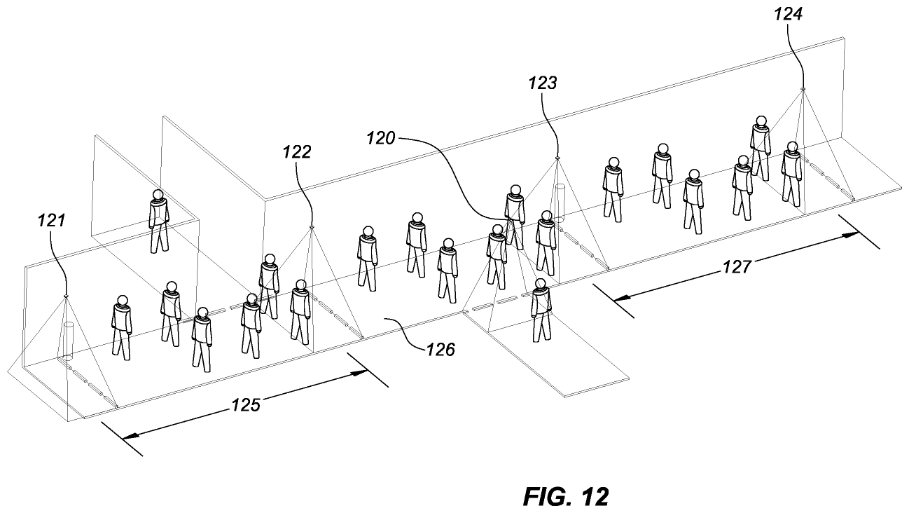

FIG. 12 shows a set of networked sensors and displays in a large occupied space. The space is divided into zones 125, 126, and 127 with occupancy sensors 121, 122, 123, 124, and 120 located at the interfaces between zones. The zones in FIG. 12 are shown as a rectangular-shaped zones on a one-dimensional grid. Alternately, the zones can be on a two-or three-dimensional grid. Alternately, the zones can be square, rectangular, or any other suitable shape. The zones are occupied as shown. The occupants can move freely between zones without their knowledge. The exemplary algorithm of FIG. 11 can be applied to this configuration to compute infection risk.

One or more of a variety of different algorithms may be used in the operation of the simulation system. An exemplary algorithm 200 is shown in FIG. 11. The algorithm 200 uses the actual or simulated historical room occupancy count in combination with the actual or simulated sensor inputs described above as well as actual or simulated air flow rates into and out of the room to compute an instantaneous pathogen infection risk in each zone. The blocks of the algorithm 200 can be performed in the order described and/or shown in FIG. 11, or in any other suitable order that is not precluded by the description below or FIG. 11.

The first steps of the algorithm may be characterized as setup of the simulation system 90, and may be performed a single time when the simulation system 90 is installed in or placed in a room 80 or other location. During setup, one or more generally-fixed physical quantities associated with the room 80 may be input into the simulation system 90. In block 202, a user inputs the dimensions of the room 80, and if the room 80 is divided into zones, also inputs the dimensions and locations of zones, such as zones 87 and 88. The user may input the dimensions of the room 80, and the simulation system 90 calculates the volume of the room 80 (and the zones in the room 80, if any) from its dimensions. According to other embodiments, the user may input the volume of the room 80 rather than the dimensions. According to other embodiments, the simulation system 90 may include a laser, lidar, radar or other sensor capable of determining the dimensions of the room 80, such that the simulation system 90 is able to determine the dimensions of the room 80 itself, and then calculate the volume of the room 80. Optionally, in block 204, the simulation system 90 may divide the zones 87, 88 into air cells. An air cell is an arbitrary volume in the room 80 or zone 87, 88 that is created for accuracy in computation. By dividing the room 80 or zone 87, 88 into a number of smaller arbitrary air cells, air flow computation can be performed in the manner of finite element analysis for simplicity and accuracy.

In block 206, the user inputs into the simulation system 90 air flow locations and rates. Referring also to FIG. 8, the air flow locations include the locations of the air supply duct or ducts 85, and the location of the return air duct or ducts 86. The flow rate at the air supply duct or ducts 85 may be controlled or measured, as may the flow rate at the return air duct or ducts 86. Optionally, in block 208, where the simulation system 90 has previously divided the zones 87, 88 into air cells, the simulation system 90 creates a matrix of air flows for each air cell in each zone.

In block 210, the user inputs into the simulation system 90 the locations and air flow rates of air disinfection systems 89. Optionally, the user inputs the type of air disinfection system 89 as well. The user may also input any other information useful to the simulation system.

The setup of the simulation system 90 is then complete. Operation may begin at block 228, in which the simulation system 90 receives input from the occupancy sensor or sensors 92 in the room 80 and/or one or more zones 87, 88 in the room 80. The simulation system 90 converts the input from the occupancy sensors 92 into a number of people in the room 80 and/or one or more zones 87, 88 in the room 80. In block 212, the simulation system 90 updates the occupancy data for the room 80 and/or one or more zones 87, 88 in the room 80, if the number of people in the room 80 and/or one or more zones 87, 88 in the room 80 has changed.

In block 214, the simulation system 90 updates the air flow rate for each cell. In block 216, the simulation system 90 calculates a decrease in pathogens in each cell, based on (a) the known settling rate of pathogens in air, (b) the rate of disinfection of the air, and (c) any other relevant factors that are sensed or calculated.

In block 218, the simulation system 90 calculates the amount of pathogens exhaled in the room 80 and/or one or more zones 87, 88 in the room 80, based on the number of occupants sensed to be located therein. The breathing rate and volume for an average person is known to be 6-8 liters/minute. The density of pathogens such as SARS-CoV-2 in air is known, such as described in Birgand et. al., “Assessment of Air Contamination by SARS-CoV-2 in Hospital Settings,” JAMA Network Open. 2020;3(12):e2033232, doi:10.1001/jamanetworkopen.2020.33232 The algorithm 200 may compute the pathogen exhalation rate based on one of three assumptions:

Assumption 1 assumes a statistical average incidence of the disease per person, based on data from the subject facility or area or state or country. This rate can be automatically updated based on known data.

Assumption 2 assumes that everyone who enters the room may have COVID-19 or the disease or diseases in question, and assumes a reference exhaled level of the pathogen for each occupant, for example 1,034 live COVID-19 virus particles per cubic meter, which has been published in a scientific journal article.

Assumption 3 assumes a statistical average incidence of the disease per person, based on data from the maximum rate of disease transmission based on the incidence of disease measured at the subject facility or in the area or state or country. This data can be automatically updated based on known data sources.

Any of these assumptions may be selected based on local conditions or for any other reason. Once an assumption has been selected, advantageously it is used consistently over a period of time until a reason exists to change the assumption. For example, a user may utilize Assumption 2 in the early stage of an outbreak of viral disease that is not under good control, then change to Assumption 1 once the average incidence of disease per person has decreased. These assumptions allow the simulation system 90 to calculate exhaled pathogen per person per minute, in combination with the known breathing rate and volume for an average person.

In block 220, based on one of these assumptions, or a combination or an average of one or more of them, the simulation system 90 may compute a pathogen density for each air cell, zone 87, 88 and/or room 80 based on the instantaneous number of occupants in each zone, in combination with the known air flow to and from one or more HVAC systems, air flow to and from neighboring zones, and the disinfection rates from disinfection devices in each zone and the consumption of disinfected air pulled in from safe air reservoirs or outside air mixing. In block 222, the simulation system 90 divides that pathogen density by the number of occupants sensed to be in that air cell, zone 87, 88 and/or room 80 to provide the pathogen dose per current occupant in that air cell, zone 87, 88 and/or room 80. Alternately, the simulation system 90 determines the pathogen dose per current occupant in any other suitable manner. The cumulative pathogen dose per occupant can be estimated from the change in the number of occupants of the air cell, zone 87, 88 and/or room 80 over a period of time, and the cumulative amount of time those occupants have been located in the air cell, zone 87, 88 and/or room 80. Each exhaled pathogen air volume may be assumed to consist of a representative fraction of exhaled pathogen particle sizes which sink to the floor at known rates. The cumulative inhaled pathogen rate per zone occupant is then computed and summed.

In block 224, the simulation system 90 compares the summed quantity of inhaled pathogens during the time the occupants are in each zone to the infectious dose of the pathogen to provide an instantaneous pathogen infection risk level. In block 226, the simulation system 90 causes the pathogen density and risk level per air cell, zone 87, 88 and/or room 80 to be displayed to one or more users, such as on display 97 and/or remote display 146. Remote display 146 may be augmented reality glasses or other augmented reality viewer, which may provide a color heat map of zone-by-zone infection risk. Augmented reality glasses or similar displays may be employed to “see” computed high risk areas in near real time. In another aspect of the invention, feedback from a surface disinfection device such as a smart sponge may be integrated for more comprehensive pathogen measurement. In addition to displaying the pathogen density and risk level in block 224, the simulation system 90 also may transmit pathogen density and risk level to an internal database, to public health authorities, to a website, and/or to any other suitable data storage hardware. As described below, such stored data may be useful for statistical, compliance, and/or other purposes.

In block 228, based on the pathogen level and risk level in the air cell, zone 87, 88 and/or room 80, the simulation system 90 may control the air disinfection system 89 in some manner. Where the pathogen level and risk level are relatively high, the simulation system 90 may actuate the air disinfection system 89 to a powered-on condition, or may increase the volume of air treated by the air disinfection system 89, or may in some other manner increase the disinfection rate of the air disinfection system 89. Where the pathogen level and risk level are relatively low, the simulation system 90 may actuate the air disinfection system 89 to a powered-off condition, or may decrease the volume of air treated by the air disinfection system 89, or may in some other manner decrease the disinfection rate of the air disinfection system 89.

Next, the algorithm iterates back to block 228, where the simulation system 90 polls the occupancy sensors 89 to determine how many occupants are located in the air cell, zone 87, 88 and/or room 80. This iteration may be performed once per defined length of time. That length of time may be a second, less than a second, less than a minute, a minute, or any other suitable time period. Alternately, the algorithm 200 does not iteratively monitor room or zone occupancy; rather, the monitoring may be interrupt-driven, such that a monitoring event occurs when a person enters or leaves the room 80, or a different or additional item that is monitored by the algorithm 200 changes.

To provide a sensitivity analysis of the simulation results, the infectious dose may be varied in the model, the average breathing rate of the occupants may be varied, and the density of viral load in exhaled breath may be varied.

Statistical techniques may be used to optimize the results of the simulation. Response surface methodologies combined with known numbers of infections may identify the most sensitive variables in the simulation and enable their optimization. Machine learning techniques may also be applied based on a set of historical simulations and known or simulated data on infection transmission in the room.

### Disinfecting Room Light System

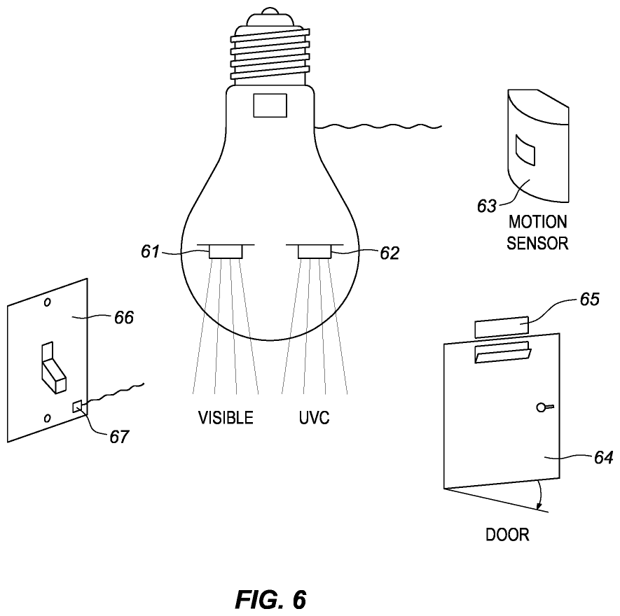

Another aspect of the invention is a programmable disinfecting room lighting system shown in the block diagram in FIG. 6. This system is intended to replace the light bulb in an existing room lighting fixture and to replace the existing wall switch. Disinfecting light system 60 combines a visible light source 61 with a germicidal light source 62 with occupancy sensor 63 and wireless (alternately, wired) sensor 64 on room door 65. Preferably, light source 61 is a standard white room light LED. Preferably, germicidal light source 62 is a UVC emitting lamp. Alternately, germicidal light source 62 is a UVC LED. Alternately, germicidal light source 62 is a far UV light source. Occupancy sensor 63 is preferably an infrared sensor. Alternately, it may be any other suitable type of sensor.

The wall light switch 66 is equipped with a wireless transmitter 67 to communicate with the lamp. Alternately, light switch 66 or other controller sends signals through the lamp power cable to the lamp for communication. By sensing if the door is closed with door sensor 64, and if the room is occupied with motion or occupancy sensor 63, the system can be programmed to only activate the UV light when the room is unoccupied and the door is closed.

In another aspect of the invention (not shown), a combination light bulb combines a far UV light source such as a lamp or LED with a visible light LED lamp into a single light source. Since the 207-220 nm far UV light inactivates pathogens without causing harm to human epithelial cells or cornea cells, the pathogen killing light may remain on even when people are in the room. Preferably, the combination of the visible light source and the far UV light source are mounted on a standard light bulb base to enable use of existing light fixtures and building electrical wiring. Optionally, the far UV light source may remain constantly on while the visible light source is switched on and off, since the far UV kills disease agents and is not harmful to humans.

### Trackable Disinfecting Sponge

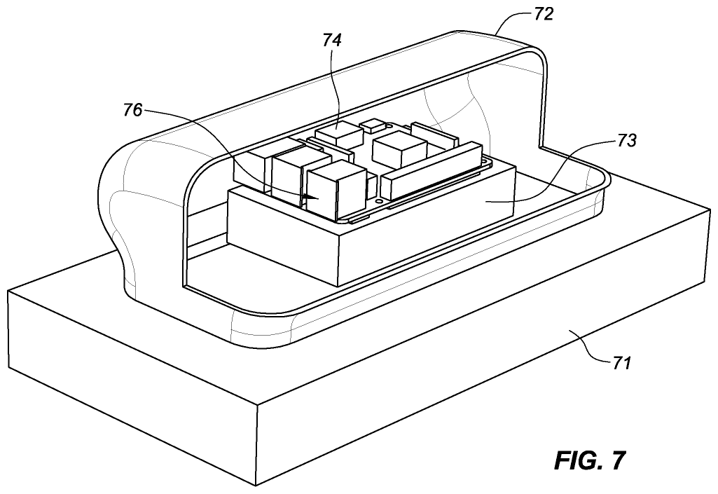

Another aspect of the present invention is a trackable disinfecting wipe or sponge 70 as shown in FIG. 7. This system is useful for fully disinfecting surfaces which may harbor pathogens. For example, the SARS-NCoV-2 virus has been shown to remain infectious on surfaces up to 17 days, thereby making it imperative that surfaces be properly disinfected. The sponge or wipe 70 consists of liquid disinfectant bearing sponge or wipe 71, waterproof housing 72 containing preferably rechargeable battery 73, microcontroller 74, with 3D accelerometer array and 3D gyroscope array 76. Optionally, electronic sponge 70 has a momentary switch (not shown) to activate the tracking system. Alternately, a motion sensor is used to turn the unit on. Optionally, a homing button (not shown) can be used to home the system when it is located over a homing label on the surface to be disinfected. The system of the present invention may alternately be used with a handle as a mop rather than a hand sponge or wipe.

Data from 3D accelerometer array and 3D gyroscope array 76 may be converted to digital signals by a set of analog to digital converters. The resulting digital signal may be filtered by an extended Kalman filter or other digital signal processing technique to remove noise. The speed of the sponge over the surface is preferably obtained by integrating the signal data from the array of 3D accelerometers and array of 3D gyroscopes 76 in sponge 70. The instantaneous or averaged speed of the sponge across the surface is used in combination with the known disinfectant exposure time based on a known pathogen kill rate to achieve a disinfection efficacy. The computed pathogen kill rate is preferably displayed by activating the appropriate color LED corresponding to the kill rate.

Alternately, a color heat map display is used to show the areas on the surface where the sponge has wiped, where the color corresponds to the speed over the surface. Alternately, or in addition, since high disinfection efficacy is inversely related to the time that the disinfection has spent on the surface, a time elapsed color map is displayed on a heat map display of the surface. Optionally, the system can link through Bluetooth or another wireless protocol to display the disinfection color heat map or instantaneous disinfection efficacy on a smart phone or a computer tablet.

Alternately, the system may incorporate an RBG-D camera for surface tracking. Alternately, the system may use a surface tracking LED/phototransistor pair for surface tracking. Alternately, a Vertical Cavity Surface Effect Laser (VCSEL) sensor is used for surface tracking. Alternately, ultra wide band transceivers are used for surface tracking. Alternately, GPS signals are used for tracking. Alternately, the system is tracked by triangulation from multiple wireless sources using time difference of arrival or other techniques. Alternately another suitable tracking technique may be used.

Disinfection system 70 may use a wide variety of cleaners and disinfectants. Exemplary disinfectants include alkyl dimethyl benzyl ammonium chloride (also known as N-alkyl-N-benzyl-N,N-dimethyl ammonium chloride, ADBAC, BC50, BC80, quaternary ammonium compounds, or quats), hydrogen peroxide solutions, chlorine dioxide solutions, sodium hypochlorite solutions, glutaraldehyde solutions, and many others. Exemplary quaternary ammonium compounds include n-alkyl (68% C12, 32% C16) dimethyl ethylbenzyl ammonium chloride and n-alkyl (60% C14, 30% C16, 5% C12, 5% C16) dimethyl benzyl ammonium chloride.

As used in this document, both in the description and in the claims, and as customarily used in the art, the words “substantially,” “approximately,” and similar terms of approximation are used to account for manufacturing tolerances, manufacturing variations, manufacturing imprecisions, and measurement inaccuracy and imprecision that are inescapable parts of fabricating and operating any mechanism or structure in the physical world.

While the invention has been described in detail, it will be apparent to one skilled in the art that various changes and modifications can be made and equivalents employed, without departing from the present invention. It is to be understood that the invention is not limited to the details of construction, the arrangements of components, and/or the method set forth in the above description or illustrated in the drawings. Statements in the abstract of this document, and any summary statements in this document, are merely exemplary; they are not, and cannot be interpreted as, limiting the scope of the claims. Further, the figures are merely exemplary and not limiting. Topical headings and subheadings are for the convenience of the reader only. They should not and cannot be construed to have any substantive significance, meaning or interpretation, and should not and cannot be deemed to indicate that all of the information relating to any particular topic is to be found under or limited to any particular heading or subheading. Therefore, the invention is not to be restricted or limited except in accordance with the following claims and their legal equivalents.

What is claimed is:

1. A method for determining pathogen risk in near real-time in an indoor area that may hold at least one occupant, comprising:
providing a pathogen risk simulation system;
inputting at least one generally-fixed physical quantity associated with the indoor area to said pathogen risk simulation system;
sensing, with said pathogen risk simulation system, at least one quantity associated with the indoor area; and
based on said inputting and said sensing, computing pathogen density and risk level in the indoor area.

2. The method of claim 1, further comprising repeating said sensing and said computing at successive intervals across a period of time.

3. The method of claim 2, wherein said intervals are substantially one second in duration.

4. The method of claim 1, further comprising controlling an air disinfection system based on said computed pathogen density and risk level.

5. The method of claim 1, further comprising displaying said computed pathogen density and risk level on a screen.

6. The method of claim 1, further comprising displaying said computed pathogen density and risk level in augmented reality glasses.

7. The method of claim 1, wherein said at least one generally-fixed physical quantity associated with the indoor area comprises the physical dimensions of the indoor area.

8. The method of claim 1, wherein said at least one quantity associated with the indoor area comprises a number of occupants of the indoor area.

9. The method of claim 1, wherein said at least one quantity associated with the indoor area comprises flow rate through an air supply duct into the indoor area.

10. The method of claim 1, wherein said at least one quantity associated with the indoor area comprises flow rate through a return air duct out of the indoor area.

11. The method of claim 1, wherein said at least one quantity is selected from the group consisting of: temperature of the indoor area, and humidity of the indoor area.

12. The method of claim 1, further comprising controlling an HVAC unit in response to said computed pathogen density and risk level

13. The method of claim 1, further comprising controlling an air disinfection system in response to said computed pathogen density and risk level.

14. A method for determining pathogen risk in an indoor area, comprising:
activating a pathogen risk simulation system;
activating sensors in the indoor area associated with said pathogen risk simulation system;
receiving input from said sensors at said pathogen risk simulation system;
based on said inputs, determining pathogen risk within the indoor area with said pathogen risk simulation system; and
communicating said pathogen risk, with said pathogen risk simulation system, to one or more users.

15. The method of claim 14, further comprising
providing an air disinfection system in communication with said pathogen risk simulation system, and
based on said pathogen risk, activating at least one disinfection system with said pathogen risk simulation system.

16. A simulation system for determining pathogen risk in an indoor area, comprising:
a central processing unit;
at least one occupancy sensor connected to said central processing unit;
at least one air disinfection system interface connected to said central processing unit; and
a display connected to said central processing unit.

17. The system of claim 16, further comprising at least one sensor selected from the group consisting of a temperature sensor and a humidity sensor, wherein said at least one sensor is connected to said central processing unit.

18. The system of claim 16, further comprising a battery connected to said central processing unit.

19. The system of claim 16, further comprising an HVAC system interface connected to said central processing unit.

20. A simulation system for controlling pathogen risk in an indoor area, comprising:
a central processing unit;
at least one occupancy sensor connected to said central processing unit;
at least one air disinfection system interface connected to said central processing unit; and
a display connected to said central processing unit.
